(19) United States
(12) Patent Application Publication
(10) Pub. No.: US 2005/0064584 A1
(43) Pub. Date: Mar. 24, 2005

(54) Cell culture vessel for the automated processing of cell cultures

(76) Inventor: Adrian Neil Bargh, London (GB)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/942,027

(22) Filed: Sep. 16, 2004

(30) Foreign Application Priority Data
Sep. 19, 2003 (EP) 03255896.7

(51) Int. Cl. C12M 1/24

(52) U.S. Cl. 435/288.1; 435/304.2

(57) ABSTRACT

The invention also relates to a cell culture vessel and in particular to a cell culture vessel assembly which aids aeration and allows for reading of the optical density of the culture without removing the culture from the vessel. The cell culture vessel assembly is suitable for use in the production and purification of cell culture products and in particular to the automated production and purification of protein.

### BACKGROUND OF THE INVENTION

The present invention relates to a cell culture vessel and in particular to a cell culture vessel assembly which aids aeration and allows for reading of the optical density of the culture without removing the culture from the vessel. The cell culture vessel assembly is suitable for use in the production and purification of cell culture products and in particular to the automated production and purification of protein.

The production and purification of specific proteins from cloned genes are essential first steps in many areas of research and development in the pharmaceutical industry. Generally, the protein of interest (or target protein) is produced within, or secreted by, cultured cells or host organisms and the target protein is recovered from the culture fluid or the cells themselves. More specifically, the gene for the target protein is linked to the appropriate DNA elements controlling transcription and translation in the host organism or cells using standard recombinant DNA techniques. During the growth of the recombinant organism or cells in the correct physical and chemical environment to trigger transcription and translation of the cloned gene, the target protein is produced. Typical host organisms and cell types that might be used in this process include bacteria such as E. coli, yeast and insect cells.

A common problem experienced with this method of producing protein is that the genes required for the production of the target proteins are not generally native to the host organism or cells used. Not only are the genes from a different species to the host organism or cells, the target proteins are often only found in certain specialised cell types. A result of this is that the host organism or cells used may comprise a non-optimal environment for the production, stability, and proper folding of the target protein.

Extensive efforts must thus be made to find appropriate culture conditions for individual strains of the host cell types used and to address nuances of modification of the gene structure in order to facilitate the production of sufficient amounts of the target protein in the desired form. A further problem experienced is that variation in the dynamics of the expression of the target protein, i.e. the rate of production and the point in the growth cycle at which expression is initiated, can have a major impact on the quality and quantity of target protein produced. To identify appropriate conditions requires the evaluation of hundreds and sometimes thousands of combinations of variables.

Methods to identify appropriate conditions for protein production are currently carried out manually or in a semi-automated fashion. Such methods, however, are slow and involve challenging experimental schedules including frequent growth monitoring, which of course is difficult to marry with normal working practices.

Furthermore, there is a limitation to the number of experiments that can be carried out in parallel and variations in operational procedures often occur creating inconsistent and non-reproducible results. An impact of the labile nature of the desired target proteins is that such variations may substantially affect the quality of the final product. There is also a health risk to staff when carrying out such large numbers of experiments such as RSI, fatigue, and exposure to genetically modified organisms, for example.

Process steps in the production of protein which have to date made it difficult to carry on its production in a fully automated fashion include measurement of optical density of the bacterial cultures. Optical density measurements of the culture must be taken at various stages in the production process in order to determine the growth stage of the cells in the culture. However, in conventional culture vessels, a sample of the culture must be removed from the vessel and diluted to get an accurate reading due to the narrow dynamic range of measuring equipment relative to changes in the density of the culture.

U.S. Pat. No. 4,105,415 discloses a test tube comprising a plurality of vertical, transparent compartments at the bottom of the tube enabling light to travel through the solution held in the test tube allowing the tester to read changes therein. FIG. 6 shows an enlarged right side section view of the bottom of the tube showing the full thickness of the tube and the narrowed portion at the lower end of the tube. This narrow compartment extends through the whole depth of the tube thus creating a confined space in the lower portion of the tube. This portion is so small and is also right at the bottom of the tube so that it is difficult to access the sediment settling in that compartment whether as a result of settling over time or whether as a result of centrifuging. Sediment collected in this part of the tube may interfere with the process of light path measurement.

In most laboratories, the use of disposable injection moulded labware is more cost effective than washing glass labware. The outer profile of the tube of U.S. Pat. No. 4,105,415 is unaltered by the taper on the internal shape and does not lend itself to injection moulding due to the large changes in wall cross-section because substantial changes in wall thickness cause mould flow problems at the time of moulding. Also, the relatively thick wall will cause distortion, commonly in the form of sink marks, during cooling from the injection moulding process and therefore adversely affect the optical characteristics of the vessel.

Culture conditions are important for correct growth of cells and the ideal conditions enable aerobic growth rather than anaerobic growth. A high level of agitation of the culture is required to maintain aerobic conditions. The profile of the tube of U.S. Pat. No. 4,105,415 significantly reduces fluid flow in the lower part of the vessel and, as a result, anaerobic growth conditions in the reduced width portion are more likely.

### SUMMARY OF THE INVENTION

According to the present invention, there is provided a cell culture vessel generally in the form of a tube having a central longitudinal axis, an open end for receiving liquid media and a closed end, the tube defining a first light path and a second longer light path, the first and second light paths being generally perpendicular to the longitudinal axis of the tube and wherein the recessed portions of the tube defining the first and/or second light path are on one side of the tube.

This configuration overcomes the difference in light paths encountered in U.S. Pat. No. 4,105,415 as the outer profile of the tube is modified by the recessed portions and therefore the light passes through the same thickness of tube material for all readings. It also allows a good fluid flow through the entire vessel.

Preferably, the tube is of a transparent material, for example, polycarbonate or is at least of a semi-transparent or translucent material, for example, polystyrene so as to allow light to pass through the vessel. The passage of light across the vessel allows for measurement of the OD of the liquid media to be taken externally of the cell culture vessel.

Preferably, the first light path is a recessed portion of the tube compared to the second light path. The first and second light paths may be recessed and non-recessed portions of the tube respectively. The first light path which is shorter than the second light path allows for the sensitive measurement of OD values when the OD values of the culture in the vessel are at a high level. The second path allows for the sensitive measurement of OD values when the OD values of the culture in the vessel are at a lower level.

Thus, the first and second light paths can be described as being defined respectively by a first tapered portion of the tube having a narrower cross-section than the tube and which tapers towards a second tapered portion of the tube having an even narrower cross section which, in turn, tapers towards the end of the tube. The “tapering” is usually when at least two opposing sides of the tube have recessed portions.

In this text, the term “recess” includes a stepped change and a gradual change.

The close end of the tube may be substantially hemispherical in shape. Preferably, the recessed or tapered portions taper towards the hemispherical closed end, directing the cells away from the narrower recessed or tapered portions towards the broader hemispherical closed end. This aids resuspension of cells which may settle at the bottom of the vessel.

According to a further aspect of the present invention, there is provided a culture vessel assembly comprising two or more culture vessels as described above. The culture vessel assembly may comprise any number of the culture vessels, such as 4, 12, 18, 24 etc. which may comprise a block of culture vessels. The culture vessels may be identical in shape and size. They may be in a unitary form (i.e. a single unit) or individual units. The unitary block of culture vessels can be formed, for example, by injection moulding.

Preferably, the culture vessel block assembly further comprises a lid for covering an open end of the culture vessel block, the lid having liquid media inlets, each of which are in register with a corresponding open end of a culture vessel of the culture vessel block.

The culture vessel block assembly may include a lip extending in a direction perpendicular to the longitudinal axes of the culture vessels and about the periphery of the culture vessel block adjacent the open end of the culture vessels for engagement with the lid. The lid can be attached to the ledge by a conventional screw to keep the culture vessel block assembly together.

Alternatively, the lid may have arms extending therefrom and generally perpendicular to the plane of the lid to engage complimentary lugs about the periphery of the cell culture block and intermediate the open and closed ends.

The culture vessel block assembly may include a perforated seal sandwiched between the lid and the open end of the culture vessel block. Preferably the seal is a sheet of resilient material, for example, rubber. When the culture block assembly is put together, the perforations of the seal are in register with the open ends of the culture vessels and the liquid media inlets of the lid to allow the passage of liquid media into the cell culture vessels.

### BRIEF DESCRIPTION OF THE DRAWINGS

The invention will be more clearly understood by way of description of an embodiment thereof given by way of example only with reference to the accompanying drawings in which:—

FIG. 1 is a perspective view and from above of an embodiment of culture aeration assembly (shown here without the upper lid portion) and culture vessel assembly according to the present invention wherein the first and second light paths are recessed and non-recessed portions of the culture vessel respectively;

FIG. 2 is a side view of the culture aeration assembly and culture vessel assembly of FIG. 1 showing mixing rods; 35: FIG. 3 is a side view of the culture aeration assembly and culture vessel assembly of FIG. 1;

FIG. 4 is a perspective view of an alternative embodiment of the invention wherein the first and second light paths are provided by two recessed portions of the culture vessel;

FIG. 5 is a cross-sectional side view of the alternative embodiment of the culture aeration assembly and culture vessel assembly clearly showing the upper lid portion of the culture aeration assembly and wherein the first and second light paths of the culture vessel assembly are tapered sections of the culture vessels; and

FIG. 6 is an end view of the culture vessel assembly of FIG. 4.

### DETAILED DESCRIPTION OF THE PRESENT INVENTION

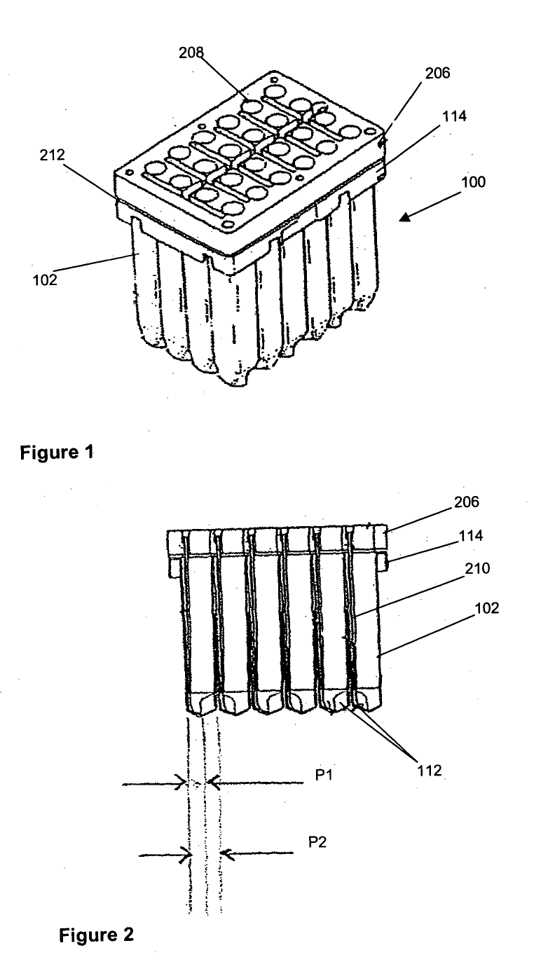

Referring now to FIGS. 1 to 3 and initially to FIG. 1, there is shown an embodiment of cell culture vessel assembly 100. The cell culture vessel assembly which is formed by injection moulding comprises a substantially rectangular shaped block 101 of 24 identical culture vessels 102 having an open end 104 and a closed end 106.

Each culture vessel 102 is generally in the shape of a rectangular cylinder having an open end 108 and a round closed end 110. The open and closed ends 108, 110 correspond to the open and closed ends 104, 106 of the culture vessel block 101. Each culture vessel 102 has a recessed portion 112 at the closed end 110. The recessed portion 112 generally extends from one side wall of the vessel 102 to midway towards an opposing side wall. This is shown most clearly in FIG. 2. The recessed portion 112 provides a light path length P1 across the culture vessel 102 for sensitive measurement of the OD of the culture when the OD of the culture in the vessel 102 is at a high level.

A light path length P2 of the non-recessed portion of the culture vessel 102 provides a longer light pathway to allow for the sensitive measurement of the OD of the culture when the OD values are at a lower level. In this way, sensitive OD measurements can be taken externally of each culture vessel 102 in situations where the culture has a high or low OD.

The culture vessel assembly 100 includes a lip 114 which extends about the periphery of the culture vessel block 101 at the open end 104 for engagement with a culture aeration assembly 200 as described in our co-pending application no. 04253377.8.

The culture aeration assembly 200, comprises a lid 202 having an upper lid portion 204 and a lower lid portion 206. This is shown in FIG. 1 where the upper lid portion 204 is not shown.

The upper and lower lid portions 204, 206 have liquid media inlets 208 corresponding to each culture vessel 102, the lower lid portion 206 having mixing rods 210 attached thereto and extending perpendicularly from the lower lid 206, and air inlets 213 for introducing air into the vessels 102. The mixing rods 210 have a first end 209 which extends into the culture vessel 102 and a second end 211 which protrudes from the top surface of the lower lid 206. The culture aeration assembly 200 includes a rubber seal 212 which has perforations 214 to receive the mixing rods 210.

To assemble the culture vessel assembly 100 and culture aeration assembly 200, the seal 212 is positioned intermediate the open end 104 of the culture vessel block 101 and the lower lid 206 of the culture aeration assembly 200 so that the seal 212 is sandwiched between the culture vessel block 101 and the lower lid 206. The culture vessel block 101, lower lid 206 and seal 212 may be held together by conventional screws (not shown). Alternatively, the lower lid 206 may have securing arms 205 which extend to engage lugs 107 on the culture vessel block 101.

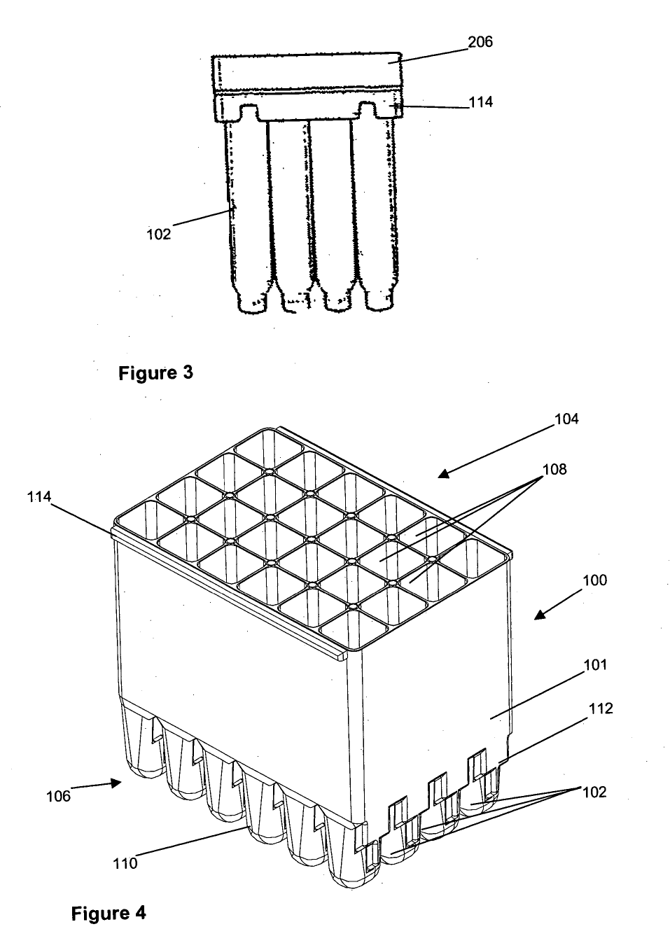

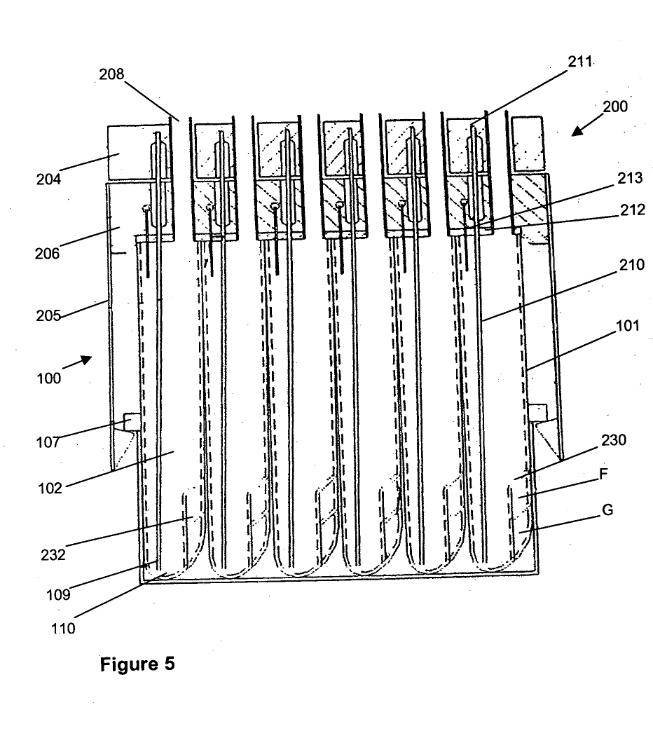

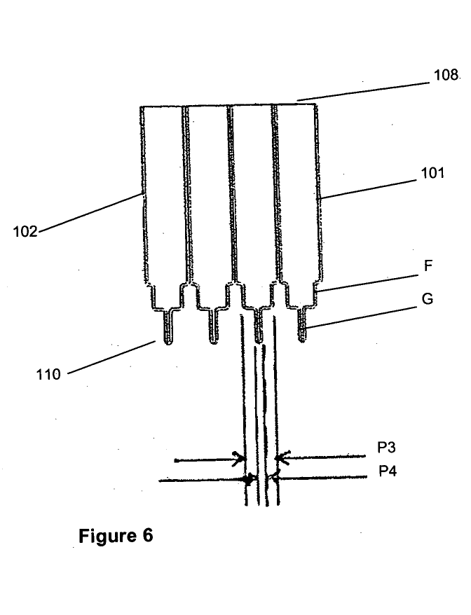

Referring now to FIGS. 4 to 6, an alternative embodiment of culture vessel block 101 will now be described where similar features are referred to by the same reference numerals.

In this embodiment of culture vessel assembly 100, each culture vessel 102 has a pair of opposing side wall portions which taper at one end remote from the open end 108 of the culture vessel 102 and in the direction of the closed end 110 to form a first tapered portion F having a light path length P3. The first tapered portion further tapers in the direction of the closed end 110 of the vessel 102 to form a second tapered portion G having a shorter light path length P4.

The light path lengths P3 and P4 provide for the sensitive measurement of the OD of the culture when the OD is at a low and high level respectively. In this manner the path lengths P3 and P4 function in a similar fashion to the path lengths P2 and P1 of the recessed culture vessel 102 described earlier and shown in FIG. 2.

The tapered portions F and G have ledges 230 and 232 respectively which slope towards the closed end 110 of the vessel 102. The ledges 230 and 232 direct settling cells to the broader section of the untapered, substantially hemispherical shaped closed end 110 of the vessel 102. This is quite clearly shown in FIG. 13 where the arrows E indicate the direction in which the falling cells are directed towards the closed end 110 of the vessel 102 by the ledges 230 and 232.

The natural rate at which cells settle to the bottom of the vessel 102 increases when the vessel 102 is centrifuged. Directing these cells to the hemispherical shaped closed end 110 of the vessel 102 and away from the narrow tapered portions F and G, aids resuspension of the cells by the action of the mixing rod 210 when the step of centrifuging is completed.

1. A culture vessel generally in the form of a tube having a central longitudinal axis, an open end for receiving liquid media and a closed end, the tube defining a first light path and a second longer light path, the first and second light paths being generally perpendicular to the longitudinal axis of the tube and wherein recessed portions of the tube defining the first and/or second light paths are on one side of the tube.

2. A culture vessel as claimed in claim 1, wherein the tube is of a transparent material, for example, polycarbonate.

3. A culture vessel as claimed in claim 1, wherein the first light path is a recessed portion of the tube compared to the second light path.

4. A culture vessel as claimed in claim 1, wherein the first and second light paths are parallel.

5. A culture vessel as claimed in claim 1, wherein the culture vessel has means for directing culture cells away from the first and second light paths.

6. A culture vessel as claimed in claim 4, wherein said means is a ledge sloping from the first and second light paths towards the closed end of the vessel.

7. A culture vessel assembly comprising two or more culture vessels as claimed in claim 1.

8. A culture vessel assembly as claimed in claim 6, wherein the culture vessel assembly comprises a unitary block of culture vessels.
